(19) United States
(12) Patent Application Publication
(10) Pub. No.: US 2004/0015073 A1
(43) Pub. Date: Jan. 22, 2004

(54) Target repositioning error correction filter and method

(76) Inventors: Michael Schell, Pittsford, NY (US); Walter O'Dell, Ontario, NY (US); Paul Okunieff, Penfield, NY (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/421,975

(22) Filed: Apr. 23, 2003

Related U.S. Application Data
(60) Provisional application No. 60/376,296, filed on Apr. 26, 2002.

(51) Int. Cl. A61B 6/00

(52) U.S. Cl. 600/411; 128/920; 378/65

(57) ABSTRACT

Disclosed are methods and apparatus for filtering an external radiation beam to create a radiation exit profile that compensates for target motion. One aspect of the invention involves a method for correcting an intended radiation dose distribution for a target, including determining a target position variance for the target, producing a three-dimensional distribution model of the target position variance, and performing an inverse blurring operation on the intended dose distribution based on the three-dimensional distribution model to produce a corrected radiation dose distribution.

### BACKGROUND OF THE INVENTION

1. Field of the Invention

This invention relates to radiation therapy techniques. More particularly, this invention relates to a method and system for correcting or filtering an external radiation beam to create a radiation exit profile that compensates for target motion.

2. Description of the Related Art

According to American Cancer Society statistics, lung cancer will account for the second greatest number of new cancer cases for both men and women this year (followed only by prostate cancer in men and breast cancer in women). Lung cancer has surpassed breast cancer as the leading cause of cancer death in women and is expected to account for 25% of all female cancer deaths in 2002. It is estimated that 85% of all new lung cancer patients will die from their cancer. The lungs are also the second most frequent site of metastatic disease and, in 20% of these cases, the lungs are the only site of metastases. The liver is the most prevalent site of metastasis, with autopsy series reporting a range of 30% to 70% for all patients who die of cancer. For example, colorectal cancer is among the top four cancers in both incidence and death rates. However, death is usually the result of metastases, and in 80% of these patients the liver is the only site of metastases. Similar data is available for breast cancer wherein 40% of patients with metastases eventually develop liver disease and 80% eventually develop lung disease.

Aggressive management of lung and liver metastases is likely to significantly improve the survival and quality of life for these patient groups. It has been shown that in colorectal cancer patients with isolated metastases to the liver, curative resection of the liver lesions lead to 30-40% 5-year survival and a complete freedom from disease in 30% of these patients. Unfortunately, 75-80% of all liver lesions are deemed unresectable due to their anatomic location and size and/or because of disease-associated hepatic cirrhosis, hyperbilirubinemia, and physical weakness that prevents surgical intervention. Faced with a significant risk of morbidity and mortality, patients often choose to forego surgery. Since there is currently no long-term effective chemotherapy for primary or metastatic liver cancer, there is very little that can be done for patients with unresectable liver tumors. If one takes into account the successes and failures of treatment for metastatic disease over the past 20 years, the technically sophisticated but intellectually simple concept of locally sterilizing metastatic disease using external-beam radiotherapy has the potential of outperforming many, if not all, of the advances in the field of the past two decades.

Medical equipment for radiation therapy treats cancerous tissue with high-energy radiation. The amount of radiation and its placement must be accurately controlled to ensure both that the tumor receives sufficient radiation to be destroyed, and that damage to the surrounding normal tissue is minimized. Movement on the part of the patient can alter the position of the radiation beam's target.

Radiation beam treatment is performed during periods of patient breath-holding to minimize respiratory-derived motion. However, the location of the target from one breath-hold to the next may vary typically on the order of 1-4 mm (O'Dell W, Schell M, Reynolds D, Okunieff P. Dose broadening due to target position variability during fractionated breath-held radiation therapy. Medical Physics 2002; 29(7):1430-1437). This target repositioning error results in an accumulated radiation field dose distribution that is a “blurred” version of the desired field dose distribution. To obtain the high doses needed to kill the tumor in the presence of such blurring, the overall dose rate ordinarily needs to be increased. The problem is that this causes increased radiation exposure to the surrounding tissue, increasing the risk of radiation injury-induced clinical complications.

Consequently, there exists a need for methods and structures to correct, or “inverse blur,” an intended radiation field dose distribution, in order to produce a corrected radiation dose distribution that will yield the intended dose distribution once administered to a patient with target motion, such as respiratory motion.

### SUMMARY OF THE INVENTION

One aspect of the invention involves a method of correcting an intended radiation dose distribution for a target, the method including determining a target position variance for the target; producing a three-dimensional distribution model of the target position variance; and performing an inverse blurring operation on the intended dose distribution based on the three-dimensional distribution model to produce a corrected radiation dose distribution.

In some embodiments, the inverse blurring operation comprises a deconvolution operation.

In certain arrangements, the three-dimensional distribution model of the target position variance comprises a Gaussian distribution.

In some embodiments, the deconvolution operation comprises an inverse Fourier transform operation using Fourier components of a Gaussian blurring function.

In a preferred arrangement, the deconvolution operation further comprises Weiner filtering.

In some embodiments, wherein said determining said target position variance comprises measuring a series of target positions using an imaging modality selected from the group consisting of computed tomography, magnetic resonance imaging, x-ray, ultrasound, and positron emission tomography.

One aspect of the invention involves a method of shaping a radiation filter, the method including determining a target position variance for a radiation target; producing a three-dimensional distribution model of the target position variance; performing an inverse blurring operation on the intended dose distribution based on the three-dimensional distribution model to produce a corrected radiation dose distribution; and shaping the radiation filter such that a radiation beam filtered by the radiation filter will produce substantially the corrected radiation dose distribution.

One aspect of the invention includes a radiation filter shaped according to the process described above.

In some embodiments, the radiation filter further includes a radiopaque material.

One aspect of the invention includes a radiation filter having a cross-sectional shape that defines an edge that is substantially sombrero-shaped.

Another aspect of the invention includes a radiation filter kit, including a plurality of radiation filters of different sizes, each of said radiation filters having a cross-sectional shape that defines an edge that is substantially sombrero-shaped.

### BRIEF DESCRIPTION OF THE DRAWINGS

FIG. 1A depicts a 2D slice through the center of an original target dose distribution 4.

FIGS. 1B through 1D depict original, blurred, and motion-corrected (“inverse-blurred”) dose profiles.

FIG. 2A shows dose profiles across the central slice for the spherical phantom dose distribution.

FIG. 2B shows dose volume histograms of the target for the corresponding runs of FIG. 2A.

FIG. 3A is a cross-section of a radiation filter with a sombrero-shaped cutout profile 8.

FIG. 3B is a perspective view of a disk filter.

FIG. 4 depicts a comparison of dose profiles.

FIGS. 5A and 5B show the dose volume histograms for the profiles in FIG. 4.

FIGS. 5C and 5D depict the results of Monte-Carlo runs using the deconvolved dose sombrero profile.

### DETAILED DESCRIPTION OF THE PREFERRED EMBODIMENT

Target position probability density can be modeled as a three-dimensional (3D) ellipsoidal Gaussian distribution. To exemplify one aspect of the invention, the effect of randomized target repositioning was assessed through numerical simulation using Monte Carlo analysis and through analytical computation using 3D convolution. These methods were demonstrated on a representative 2-arc, 10-fraction treatment plan that was used to treat individual tumors in a human subject presenting with multiple metastatic lung lesions. The effect of the magnitude of the position variability on the changes in the observed dose was studied using standard deviations in tumor position that ranged from 1 to 4 mm. For the same human subject, tumor position variability data was gathered from pretreatment magnetic resonance image (MRI) datasets. The measured variability values were also applied in the analysis. Using representative tumor position variability values and corresponding margin definitions, a comparison was made between the volumes of lung tissue that would be expected to receive harmful doses of radiation using end-expiration breath holding versus end-inspiration breath-holding during treatment planning and radiation delivery. Dose volume histograms (DVHs) were employed to assess exposure to both the target and the surrounding healthy tissue, as well as to compare the results across the various methods.

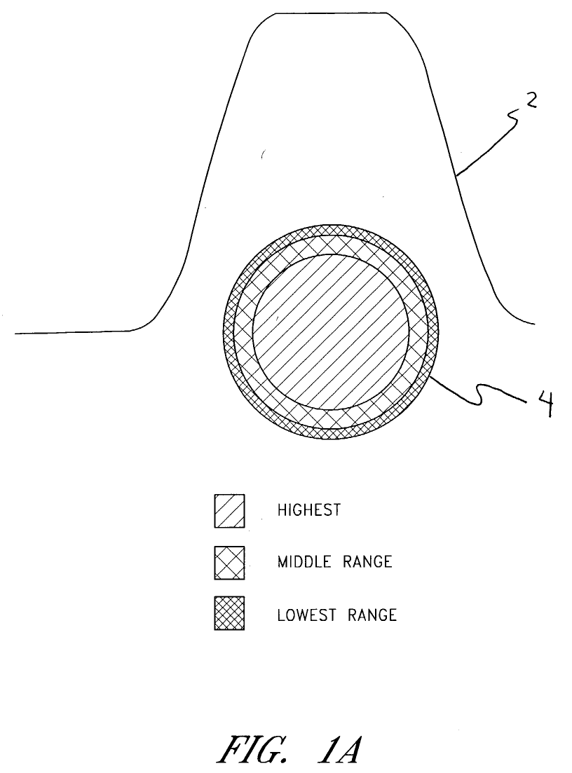

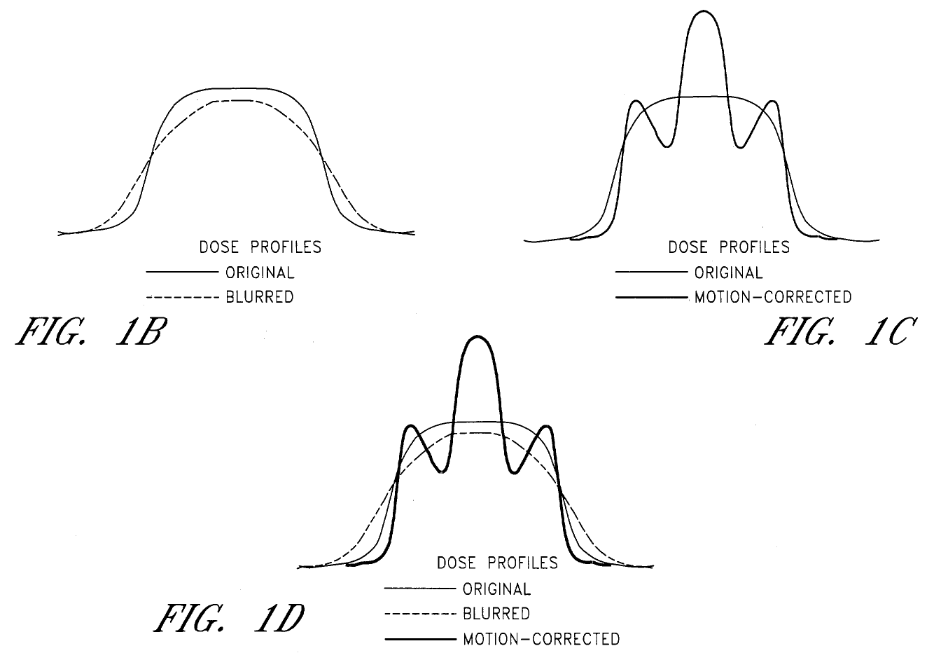

FIG. 1A depicts a 2D slice through the center of an original target dose distribution 4. The concentric circles represent the target dose distribution 4. The curve 2 overlaying the image is the dose profile through the center of the slice. FIGS. 1B through 1D depict original, blurred, and motion-corrected (“inverse-blurred”) dose profiles.

Target Position Variability Model

For fractionated radiotherapy with breath-holding, the variability in position of the target over all the repeated breath-holds was modeled as a Gaussian distribution. The standard deviation in position about the mean position was allowed to vary independently with coordinate direction, where the coordinates were defined with respect to the patient: Superior-inferior (SI); anterior-posterior (AP); and right-left (RL). The resulting 3D Gaussian ellipsoid was sampled at 1 mm intervals in each direction to create a digitized position probability matrix. It is important to sample the Gaussian probability matrix out a sufficient distance to ensure that the target position probability at the edge voxels is very small. This was achieved by sampling out to a distance equal to 2 times the Gaussian standard deviation. For the 4 mm position variability model, the Gaussian probability matrix was generated over 25×25×25 grid, where each voxel corresponds to 1 cubic millimeter.

Dose Perturbation Via Convolution

As the number of treatment fractions approaches, infinity, the cumulate dose seen by the target can be modeled as the convolution of the 3D dose distribution with the 3D Gaussian probability distribution for target position, as described previously by several authors. These include, inter alia, B. K. Lind, “Optimal radiation beam profiles considering uncertainties in beam patient alignment,” Acta Oncol. 32, 331-442 (1993); V. Rudat, “Influence of the positioning error on 3D conformal dose distributions during fractionated radiotherapy,” Radiother. Oncol. 33, 56-63 (1994); and A. E. Lujan, “A method for incorporating organ motion due to breathing into 3D dose calculations,” Med. Phys. 26, 715-720, (1999).

The convolution operator is described by the equation

$$\begin{matrix}f [ l , m , n ] = g [ l , m , n ] ⊗ h ( i , j , k ] \\ = {\sum }_{I} {\sum }_{J} {\sum }_{K} h ( i , j , k ] \cdot g [ l - i , m - j , n - k ] ,\end{matrix}$$

where g is the original (planned dose field represented as a 3D matrix (of size L×M×N) of dose values, h is the 3D Gaussian distribution model represented by a 3D matrix of size I×J×K, and f is the resulting, altered dose distribution. The values of I, J, and K increase (decrease) with increasing (decreasing) position variability in the SI, AP, and RL directions, respectively. The analyses using convolution and the generation of dose field plots and their derivatives were performed using dedicated Java code written by the authors and based on the NIH ImageJ software package (developed at the U.S. National Institutes of Health).

Target Position Variability Via Monte Carlo Simulation

The dose field perturbation for a finite number of treatment fractions can be assessed using Monte Carlo simulation, as demonstrated J. Leong, “Implementation of random positioning error in computerized radiation treatment planning systems as a result of fractionation,” Phys. Med. Biol. 32, 327-334 (1987); and others, such as J. H. Killoran, “A numerical simulation of organ motion and daily setup uncertainties: implications for radiation therapy,” Int. J. Radiat. Oncol., Biol., Phys. 37, 213-221 (1997); A. E. Lujan, “A method for incorporating organ motion due to breathing into 3D dose calculations,” Med. Phys. 26, 715-720, (1999); and M. A. Hunt, “The effect of setup uncertainties on the treatment of nasopharynx cancer,” Int. J. Radiat. Oncol., Biol., Phys. 27, 437-447 (1993).

A random-number generator was modified to produce both negative and positive output displacements that fall within a prescribed Gaussian distribution. The generator's output was used to shift the prescribed dose field in each of the SI, AP, and RL directions randomly for 10, 20, 100, or 1000 treatment fractions. The method of Monte Carlo simulation applied to a digitized dose field requires displacements of the target to be integer valued. The numbers returned from the random number generator were first scaled to fit the desired Gaussian distribution and then rounded to the nearest whole number. This analysis was performed using Microsoft Visual Basic running within Excel.

Lung Lesion Treatment Plan

A realistic treatment 3D dose field was obtained from a conventional conformal beam treatment plan for a representative lung lesion in a human subject. The selected lesion was approximately 12 mm in diameter and located posteriorly inferiorly in the patient's left lung. The treatment plan for this lesion consisted of 6-MV X rays applied in two, 110-degree arc pathways separated by 20 degrees. The clinical target volume (CTV) was defined by the observable tumor as manually segmented using the commercial planning system software. The planning target volume was defined as the CTV with a margin specification of 7×7×10 mm in the AP, RL, and SI directions, respectively. The plan was created using the BrainLAB Novalis system and BrainScan 5.0 software and implemented at the University of Rochester's R. J. Flavin Novalis Shaped Beam Surgery Center. Each arc was administered daily, during independent 15 second breath-holds. The external beam radiation was ministered daily over 10 days of treatment to give a total dose to the lesion of 50 Gy. Patient repositioning between treatment days was performed using BrainLAB's Exac Trac Patient Positioning System (EPPS). The EPPS tracks skin-affixed retro-reflective marks with a pair of infrared cameras. The Novalis unit was designed for stereotactic radiosurgery with an alignment tolerance of 0.75 mm. The EPPS positioning uncertainty is 1.5 mm.

Lung Lesion Position Variability Measurements

Prior to radiation therapy, repeated volumetric MRI datasets of the lungs were acquired in the patient volunteer. All procedures were performed in accordance with federal and university guidelines and the patient had given informed consent. The three sets of chest MRI volume data were acquired during ˜30 second periods of patient breath-hold at relaxed end-expiration. The patient volunteer was instructed to perform two deep breaths followed by a relaxed expiration and breath-hold. Each MRI 3D dataset consisted of 168 overlapping sagittal slices with slice thickness 4 mm and slice center-to-center separation of 1 mm. For each of the five lesions, the centroid was manually demarked and the mean position and standard deviations about the mean position were then computed.

End-Expiration Breath-Holding Versus Deep End-Inspiration Breath-Holding

A secondary hypothesis is that the improved target localization with end-expiration breath-holding (EEBH) and accompanying reduction in treatment margin will more than compensate for the decreased lung density but larger margins associated with deep end-inspiration breath-holding (DIBH). The current literature provides no quantification of the motion of isolated tumors in the lung and/or liver during respiration, or of tumor position reproducibility over multiple breath-holds. However, studies have been performed looking at diaphragm position during free breathing and over repeated breath-holds, suggesting that target position variability is improved with EEBH compared to DIBH. Using these values, treatment plans were constructed using conventional margin specifications to ablate the representative lung lesion. Lung density changes from end-expiration to deep inspiration were included in the estimate of the volume of surrounding lung tissue exposed to high dose in each treatment approach.

Target Position Variability and Dose Broadening

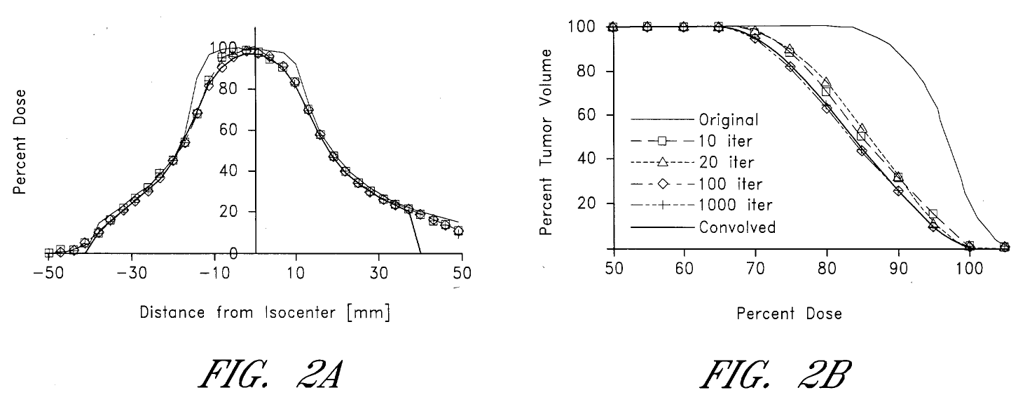

The result of the convolution of the dose matrix by the position variability matrix is an eroded version of the original dose distribution, as depicted in FIG. 2A and as described by previous authors, such as B. K. Lind, “Optimal radiation beam profiles considering uncertainties in beam patient alignment,” Acta Oncol. 32, 331-442 (1993); and J. Leong, “Implementation of random positioning error in computerized radiation treatment planning systems as a result of fractionation,” Phys. Med. Biol. 32, 327-334 (1987).

The target position variability alters the original dose distribution by increasing the dose field width at the lower doses, reducing the width at the higher doses, and decreasing the slope of the dose profile edges. FIG. 2A shows dose profiles across the central slice for the spherical phantom dose distribution for the case of a fixed target (“Original”) and target position variance of 4 mm as computed using the Monte-Carlo method with 10, 20, 100, and 1000 iterations, and using convolution. FIG. 2B shows dose volume histograms (DVH) of the target (“Tumor”) for the corresponding runs. The effect of target position variability on the cumulative dose seen by the target is depicted by the corresponding DVHs for the target. The relationship between the leftward shift of the tumor DVH curve and the magnitude of the position variability is monotonic but nonlinear, as seen quantitatively in FIG. 2B. The decrease in the dose at the 50% tumor volume at 1 mm target position standard deviation is less than 0.2%, but the decrease in dose becomes 1.7% for a standard deviation of 4 mm. The volume of surrounding tissue receiving ≧50% does and that receiving ≧80% dose dropped as the position standard deviation increased.

Finite Versus Infinite Number of Treatment Fractions

One can compare, as we did, for example, the original and shifted tumor DVHs for a 4 mm standard deviation in position computed using both the convolution equation and Monte Carlo method using single trials at 10, 20, 100, and 1000 fractions. The results for the 100-fraction and the 1000-fraction Monte Carlo runs were nearly identical, suggesting that 100 fractions are sufficient to achieve convergence for this approach. The DVH values and dose profiles for the convolution method were distinguishable from those of the 1000-fraction Monte Carlo method, determining the equivalence of the approaches. There is a statistical relationship between the expected results from any given 10-fraction simulation and the finite-fraction results (the convolution method), as described by sampling theory. Namely, the expected standard deviation of the mean, σ_{M}, for N number of fractions is given by σ_{M}²=σ_{P}²/N, where σ_{P} is variance of the total population. To better illustrate this phenomenon, 50 trials using 10 fractions were conducted using the Monte Carlo approach. From the 50 trials, the means, 25^{th} quartile, and 75^{th} quartile DVH curves, computed as percent tumor volume varying as a function of percent dose, were compiled and plotted alongside the DVH curve computed using the convolution method. This analysis was repeated for 50 runs of 20 fractions. These graphs (not shown) give the expected spread in the DVH outcomes associated with using a finite number (10 or 20) of treatment fractions. These graphs give the expected spread in the DVH outcomes associated with using a finite number (10 or 20) of treatment fractions. The randomly chosen 20-fraction trial happens to lie outside the 20-fraction quartile range, but this is not unexpected in light of the sampling theory.

Lung Lesion Position Variability Measurement and Dose Broadening

Breath holding for the duration of the MRI acquisition (˜30 seconds) was well tolerated by the patient volunteer. As shown in Table I, the average variability in position for all five lesions and over three repeated breath-holds was found to be less than 3 mm in any direction, with the greatest variability in the superior-inferior direction compared to the anterior-posterior and lateral directions. At physiologic variability values, the percent of tumor volume seeing 100% of the dose (50 Gy) was 44%, compared to 56% for a fixed target. However, in both cases all of the tumor volume observed ≧96% dose (or 48 Gy)-above the tumoricidal threshold.

End-Expiration Breath-Holding Versus End-Inspiration Breath-Holding

With breath-holding protocols, the reproducibility of the end-expiration location of the diaphragm from breath-hold to breath-hold is less than 0.4 mm, while that for end-inspiration is approximately 1.3 mm. The diaphragm also moves during a breath-hold. During a relaxed breath-hold at end-expiration (EEBH) the diaphragm displaces superiorly at a rate of 0.15 mm/s␣0.7 mm/s. This leads to an overall shift in the superior-inferior direction of 1.9 mm±1.4 mm for a 20-second breath-hold. For breath-holds at relaxed end-inspiration, the total displacement is 2.3 mm±1.2 mm with a very nonlinear velocity profile. Korin et al. also performed a preliminary study looking at the global motion of the liver. They found that the motion of the liver follows closely that of the diaphragm during relaxed breathing, with SI motion dominant, and AP and RL motions<2 mm. In comparison, Hanley et al. reported that diaphragm repositioning error for repeated deep inspiration breath-holding (D113H) was 2.5 mm and that motion during each breath-hold was 1.0 mm (standard deviation about the mean position).

These DIBH studies were performed using a spirometer to improve the reproduction of inspiration levels. A conservative plan would entail specifying a planning target volume (PTV) with treatment margins around the tumor equal to two times the expected standard deviation in position plus two times the expected motion range. First, using a back-of-the envelope comparison of planning target volumes, we consider a 12 mm diameter clinical target volume. Using the inspiration data from Balter's and Holland's papers (1.3 mm and 2.3 mm, respectively), 7.2 mm of margin would be needed. This leads to a planning target volume of 9.6 cm³. The corresponding margin for an end-expiration breath-hold, based on Balter's and Holland's results (0.4 mm and 1.9 mm, respectively), would be 4.6 mm, giving a planning target volume of 5.0 cm³. This represents a reduction in healthy tissue volume within the PTV of 4.6 cm³, or 53% using the EEBH plan compared to the DIBH plan. For an expected decrease in DIBH lung density of 26% (Ref. 24) compared with the EEBH technique, the DIBH technique would result in a less favorable lung mass exposure compared with the end-expiration breath-holding approach. For a 20 mm diameter CTV (perhaps more reasonable for this 12 mm lesion considering the inherent inaccuracies in the diagnostic imaging system and the potential for microscopic extent of the disease) the same analysis gives a reduction in healthy issue volume being irradiated of 8.3 cm³, or 48%, when using EEBH compared to DIBH. With the inclusion of dose spread effects, the results were similar. Two treatment plans were created for the representative lung lesion in the manner described earlier but with axisymmetric treatment margins of 4.6 and 7.2 mm, to represent the EEBH and DIBH plans, respectively. The treatment plan with the 4.6 mm margin was then convolved with a Gaussian target position variability model with an axisymmetric target position standard deviation of 2.3 mm. This was repeated for the 7.2 mm margins plan and a 3.6 mm standard deviation of target position.

For the EEBH case the absolute volume of surrounding lung tissue receiving ≧50% dose was 9.4 cm³. That for the DIBH values, after adjustment for the 26% decrease in lung tissue density due to increase air volume, was 12.4 cm³ (a difference of 24%). For the volume of lung tissue receiving ≧80% dose, the EEBH and DIBH methods gave 2.39 and 3.15 cm³, respectively: a difference again of 24% and very similar to the back-of-the envelope calculation result above.

Clinical Results

In regards to the measurement, modeling, and verification of the dose broadening effect, the results were as follows. A treatment margin specification of 7×7×10 mm, in the AP, RI, and SI directions, respectively, was chosen by the attending physicians. The target repositioning variability results from the repeat end-expiration MRI datasets indicate that these margins represent a size equal to or greater than three times the expected standard deviation in position. For a typical 12 mm diameter tumor, this size margin would ensure that the entire tumor remains within the high-dose region for each fraction with 99.87% certainty, assuming that sources of target position variability other than that due to breath-holding error are negligible. The results presented in this paper demonstrated that the entire tumor volume was irradiated to a minimum of 48 Gy—well above the tumoricidal threshold. This finding was substantiated by the clinical results: there was evidence of tumor shrinkage during treatment and all but one of the lesions had disappeared completely by the end of the 10-day therapy. At the 6-month and 12-month follow-ups, all five lesions had been eradicated with no indication of disease recurrence at the treatment sites.

Edge Effects

Artifacts will occur at the boundaries of the dose field when the dose does not fall to zero smoothly, such as occurs at the surface-air interface and at the edges of the sampled dose field. The size of the boundary zone where artifacts may appear is determined by the standard deviation (σ) of the target position probability distribution and for most practical purposes is limited to 3σ. In the current study, the dose field was sampled over a 10 ×10×10 cm region centered about the lung lesion. For simplicity of the calculation using the convolution and Monte Carlo methods, the dose values for locations outside the sampled dose field were assumed to be zero. However, the measured dose on the perimeter of the sampled dose field was found to be significant at some locations, with a maximum dose at the border of the sampled region of 41%. The error occurs where there is an abrupt dose profile drop-off at the outer-most 2-7 mm of the measured dose profiles. This solution to the edge artifact is to sample the dose field out into the zero-valued regions, or at least sufficiently far from the target and the lung volumes so that the edge effects do not corrupt the DVH results. The limited spatial extent of the dose field sampling also prevents an accurate determination of the lung tissue DVH values for doses less than ˜41%, although the spatial extent of the sampling was sufficient for an accurate determination of all tumor DVH values and lung tissue DVH values ≧50%. In addition, because of the relatively small size of the planning target volume compared with the entire left lung volume, 12 cm³ versus 1700 cm³, less than 2% of the left lung volume sees ≧50% dose.

Patient Surface to Radiation Source Distance Changes and Effects

Neither the Monte Carlo nor the convolution methods as used herein take into account the changes in dose as the target moves randomly toward or away from the radiation source during each fraction. However, the effects of changes in both the distance from the patient surface to the radiation source (SSD), and the changes in the depth of the target relative to the surface were studied by McCarter and Beckham, wherein they concluded that the variations in position both above and below the mean depth added to give an insignificant net effect, even for very large standard deviations (up to 100 mm). In addition, Bel et al. quantified the SSD effect to be less than 1% for a relatively large, one-time 7.5 mm shift along the beam trajectory of one beam in a 3-beam plan.

End-Expiration Breath-Holding (EEBH) Versus Deep-Inspiration Breath-Holding (DIBH)

Using end-inspiration breath-holds to compensate for respiratory-derived lung tumor motion results in a less favorable lung mass exposure in the high dose region compared to using an end-expiration breath-holding approach, even after considering the decrease in lung tissue density due to the increased volume of air in the lungs for DIBHs. This is primarily a result of the larger variability in diaphragm position over repeated breath holds at end-inspiration compared to end-expiration, even when lung volume feedback via a spirometer is used in the end-inspiration method and not in the EEBH method. End-expiration breath-holding is therefore recommended over DIBH for fractional radiation therapy of small lung and liver lesions because of improved target position reproducibility, decreased radiation exposure to the surrounding tissue, and because it can be used successfully without the aid of a spirometer or similar device that may diminish patient comfort and compliance.

Mathematical Description of Target Position Probability

A goal of subsequent research in this field is to establish the efficacy of using common statistical models, such as a Gaussian distribution, to represent tumor position variability for these lesions. A much larger pool of organ and target repositioning data must first be acquired before any statistical representation can be accepted with confidence. However, the adherence of tumor repositioning to a common statistical model is not strictly required for the application of either the convolution or the Monte Carlo approaches. These methods required only the determination of the 3D target location probability distribution.

Margin Size Versus Target Position Variability

The current study used the dose field sampled from a treatment plane using clinical margins of 7×7×10 mm (AP×R×SI). These margins are large in comparison to the expected and tested target position variability values, thus the effect of target position variability on the tumor DVH response was correspondingly small. Dose broadening studies on dose fields creating using smaller treatment margins produce larger changes in the tumor DVH response (data not presented), but the nature of the response remains the same.

Breath-holding enables us to reduce margin size such that a lethal dose can be administered to the tumor while the volume of healthy tissue receiving toxic dose is kept within clinically acceptable limits. However, radiation toxicity to the surrounding tissue remains a major concern. In our initial patient treatment protocol, a margin size of 7×7×10 mm was used for each of five lesions. For a representative 12 mm diameter lesion, 11.3 cc of total tissue volume is thereby irradiated with lethal dose, of which only 8% is the tumor. For five such lesions in a patient, a total of 52 cc of healthy tissue is destroyed, along with an equally large volume of lung tissue exposed to harmful but nonlethal levels of radiation. In otherwise healthy patients, this loss of lung tissue volume does not pose a serious health risk; however, it is conceivable that additional lung lesions may present in these patients over their lifetimes. The cumulative loss of lung volume for a large number of sites may prove clinically significant. An application of our ability to model the dose erosion effects due to target repositioning error is to improve the methods used for selecting treatment margins.

During fractionated radiotherapy, target position variability over repeated breath-holds broadens and diminishes the amplitude of the dose distribution seen by the target and surrounding tissue, compared with that seen by a fixed target. If a probabilistic model can describe the distribution of target positions, then this dose-erosion effect can be quantified using image convolution theory and Monte Carlo simulation. Using dose distributions acquired from radio-therapy treatment plans, we have shown that the changes in the dose distribution for target position variability of less than 1 mm are negligible. Using pretreatment MRI acquisitions over repeated breath-holds, we were able to estimate the expected target repositioning error for an individual subject and use this to predict the changes in the dose field that occurred during the subsequent treatment. We were then able to show that the entire tumor was subjected to a cumulative dose in excess of 47 Gy—well above the tumoricidal threshold. This information suggests that curative treatment of lung and liver lesions is possible when simple end-expiration breath holding is used to compensate for respiratory-derived motion.

Dose Field Broadening

The effect of randomized target repositioning is assessed through numerical computation using 3D convolution of the computed dose distribution with a 3D Gaussian distribution representing the known variance in each dimension (an ellipsoidal Gaussian profile in 3D). The effect of target position variance on the observed dose field was computed for the three test cases and a variety of position variances and treatment margins. The standard deviation in position about the mean position is allowed to vary independently with coordinate direction, where the coordinates are defined with respect to the patient: Superior-Inferior (SI); Anterior-Posterior (AP); and Right-Left (RL). The resulting 3D Gaussian ellipsoid is sampled at 1 mm intervals in each direction to create a digitized position variance matrix.

Since tumor position variance follows a Gaussian distribution model, then as the number of treatment fractions approaches infinity the accumulated dose seen by the target can be modeled in various mathematical ways, which are ways to express the “blurring” that occurs during fractionated radiation therapy involving target motion variance. These mathematical operations may thus be called “blurring operations.” In one embodiment, the accumulated dose seen by the target can be modeled as a convolution of the 3D dose distribution by the 3D Gaussian distribution matrix. The convolution (a blurring operation) is described by the equation

$$\begin{matrix}f [ l , m , n ] = g [ l , m , n ] ⊗ h ( i , j , k ] \\ = {\sum }_{i} {\sum }_{j} {\sum }_{k} h ( i , j , k ] \cdot g [ l - i , m - j , n - k ]\end{matrix} \qquad ( 1 )$$

where g is the original dose distribution (a 3D matrix of size L×M×N), h is the Gaussian distribution model (a 3D matrix of size I×J×K), and f is the resulting broadened dose distribution.

Our second test case consisted of the radiation treatment plan created using a commercial planning system (BrainScan 5.0, BrainLAB, Redwood City, Calif.) for a spherical test phantom (radius 11.8 mm). FIGS. 2A and 2B depicts the dose profile though the treatment isocenter before and after a simulated broadening with 4 mm target position variability and comparing both the convolution and Monte-Carlo methods.

FIG. 2A shows dose profiles across the central slice for the spherical phantom dose distribution for the case of a fixed target (“Original”) and target position variance of 4 mm as computed using the Monte-Carlo method with 10, 20, 100, and 1000 iterations, and using convolution. FIG. 2B shows a dose volume histogram (DVH) of the target for the corresponding runs.

Optimization of the Beam Profile

An equivalent method for computing the convolution of two matrices uses the Fourier transformation of the two matrices. For G defined as the Fourier transform of the original dose distribution g (refer to Equation 1), and H the Fourier transform of the blurring function h, the Fourier transform of the resulting convolution is given by straight-forward multiplication in the Fourier domain, where i, j and k are indices of the frequency components of the respective matrices:

F_{i,j,k}=G_{i,j,k}·H_{i,j,k} (2)

Any operation that is an inverse of a blurring operation is herein called an “inverse blurring operation.” For example, as multiplication of the original dose distribution's Fourier transform (G) by the Fourier transform of a Gaussian blurring function (H) results in a blurred dose distribution, the inverse operation can be used to “unblur,” or “inverse blur,” a dataset. This deconvolution approach is commonly performed to correct for motion blurring artifact in photographic images, especially prevalent in satellite image restoration.

Direct division of the resulting blurred distribution's Fourier transform components (F) by the corresponding blurring function's Fourier components (H) leads to significant artifact caused by the frequency-band-limited nature of the blurring function and the presence of noise in the measured dose distribution values (F). To alleviate most of this artifact, Wiener filtering is performed, according to the equation:

$${G}_{i , j , k} = {F}_{i , j , k} \cdot \frac{{H}_{i , j , k}}{{H}_{i , j , k}^{2} + {α}_{i , j , k}} \qquad ( 3 )$$

The elements of matrix α are the ratios of the power of the noise signal to the power in the true signal at the frequency (i,j,k). The ratio of signal powers is often not known precisely. In our implementation, the power at each frequency in the resultant, blurred dataset was first computed. A cut-off frequency was then determined after inspecting the power spectra profile. All frequency components higher than the cut-off were set to zero, and the smallest power value of the frequency components below the cut-off was set as the power of the noise in the dataset. The noise power was then assumed to be constant over all the remaining frequency components. Equation 3 effectively sets G_{i,j,k} to be nearly F_{i,j,k}/H_{i,j,k} at those frequencies where the true signal is much stronger than the noise signal, and sets G_{i,j k} to zero where the noise power dominates.

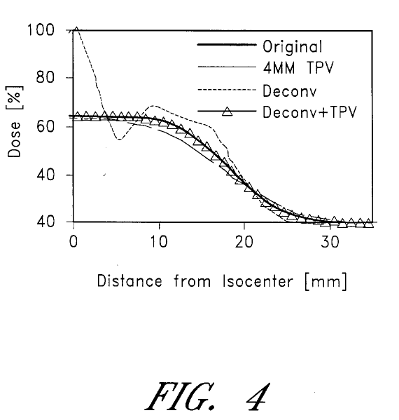

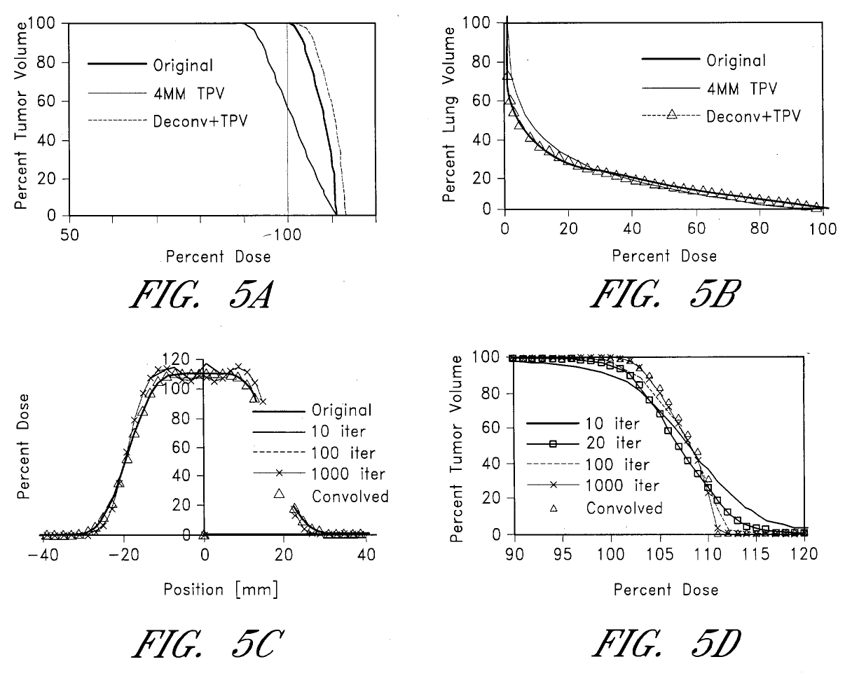

The application of deconvolution theory and Weiner filtering to compute a broadening-corrected beam profile is a powerful method for further reducing harmful clinical side-effects of radiation therapy. We have computed the deconvolution profile for the computer-generated, spherically symmetric test case and the preliminary results are shown in FIGS. 4 and 5A-D. The result of the deconvolution operation for the ideal symmetrical dose distribution is the symmetrical dose profile with a steep slope for the dose field at the field edges and a very intense peak field at the center of the target surrounded by troughs in the field on either side. This is referred to as a “sombrero” profile because of its resemblance to a Mexican hat, with a tall central elevation and two side elevations. In the presence of position variability, the resultant blur of the deconvolved dose field diminishes the slope at the periphery and flatten outs the central peak and surrounding troughs to produce a dose field that is nearly identical to the original, ideal dose field prior to blurring. A comparison of profiles is shown in FIG. 4. The DVH's for each case are compared in FIGS. 5A and 5B. The deconvolved and blurred target DVH is nearly identical to the unblurred, original target DVH except for a shift to the right of 3% dose. The lung DVH is nearly identical in both cases. In an actual treatment, a finite number (20-30) of treatment fractions are used, rather than the infinite number implicitly assumed in the convolution analysis. To gauge the effect of a finite number of target position samples on the resulting observed dose, Monte-Carlo runs were performed using the deconvolved dose sombrero profile and the results are shown in FIGS. 5C and 5D.

Intensity-Modulated Radiotherapy (IMRT)

The corrected dose profile of the invention may be administered to a patient in a variety of ways, such as by shaping a filter, as described below, especially with a sombrero-shaped cutout profile. Alternatively, one may vary the intensity of the exit beam in other ways to create the corrected, desired dose distribution. One way is by employing intensity-modulated radiotherapy (IMRT). This technique is well known to those of skill in the art, and is described in, for example, U.S. Pat. No. 6,449,336 to Kim et al., the entirety of which is incorporated by reference herein. Using IMRT, one can increase the intensity of the radiation beam where appropriate by, for example, slowing the beam over the target. And one can decrease the intensity of the radiation beam by, for example, increasing the speed of the beam over the target. In some embodiments, this selective exposure of the target using IMRT makes use of optimal and appropriate speeds, exposure times, and intensity, as determined according to principles of the invention by the dose-profile correction (“inverse blurring”) algorithms and resultant corrected dose profiles described herein.

Target Repositioning Error Correction Filter

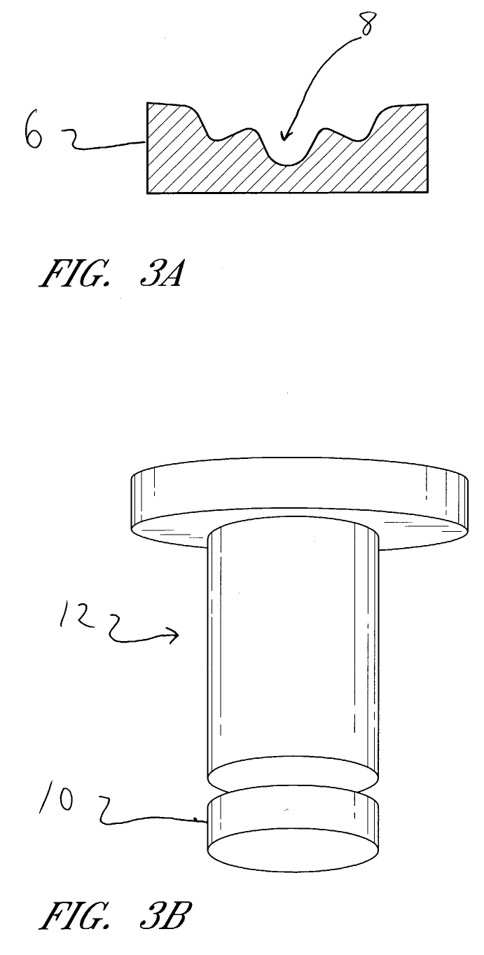

FIG. 3A is a cross-section of a radiation filter 6, displaying a sombrero-shaped cutout profile 8 (particularly if viewed upside down relative to the view shown in FIG. 3A.

FIG. 3B is an embodiment of a machine-milled disk filter 10 made of radiopaque material that can interface with existing radiation therapy hardware 12.

The advantages of a Target Repositioning Error Correction Filter (TRECOF) compared to existing technologies include: 1) the simplicity of the device, 2) enhanced 3D-treatment geometry, and 3) great accuracy and reliability.

The filter is milled into shape by a high precision-milling machine prior to the initiation of therapy and mounted on the machine for each field. This filter eliminates the need for multileaf collimation (MLC) for small lesions (<20 mm diameter). While it can be argued that an MLC has advantage of modification of field collimation without the radiation therapist transporting a device to the machine, the multileaf collimator's primary disadvantage is an extremely complex and expensive device. The MLC consists of 80 multileaf drive motors. Our past experience on a Varian CL21EX linear accelerator has been that approximately ⅓ of the motors have failed in the first year at the cost of $6,000 per motor. This machine could be used for intensity-modulated radiotherapy (IMRT). IMRT would triple the use of the motors and thereby triple the failure rate.

The second and perhaps even more significant advantage is that without an MLC, distance from the patient to collimator is increased. This allows for the machine to rotate through a wider gantry angle range. Currently, some treatment angles are prohibited by the presence of the bulky MLC collimator thereby impeding the optimal gantry angle range for 3D conformal radiation therapy.

The third advantage of the TRECOF device is that the accuracy of the milling machine allows for a beam modifier that has much higher spatial resolution than that encountered with the 5 mm wide MLC leaves or even the 3-mm wide leaves encountered on micro-multileaf collimators available today.

What is claimed is:

1. A method of correcting an intended radiation dose distribution for a target, the method comprising:
determining a target position variance for the target;
producing a three-dimensional distribution model of the target position variance; and
performing an inverse blurring operation on the intended dose distribution based on the three-dimensional distribution model to produce a corrected radiation dose distribution.

2. The method of claim 1, wherein the inverse blurring operation comprises a deconvolution operation.

3. The method of claim 1, wherein the three-dimensional distribution model of the target position variance comprises a Gaussian distribution.

4. The method of claim 1, wherein the deconvolution operation comprises an inverse Fourier transform operation using Fourier components of a Gaussian blurring function.

5. The method of claim 4, wherein the deconvolution operation further comprises Weiner filtering.

6. The method of claim 1, wherein said determining said target position variance comprises measuring a series of target positions using an imaging modality selected from the group consisting of computed tomography, magnetic resonance imaging, x-ray, ultrasound, and positron emission tomography.

7. A method of administering radiation to a patient in a corrected radiation dose distribution, the method comprising:
providing a corrected radiation dose distribution, said corrected radiation dose distribution (a) resulting from having an inverse blurring operation performed on an intended dose distribution, and (b) based on a three-dimensional distribution model of a target position variance; and
administering radiation to the patient in said corrected radiation dose distribution.

8. The method of claim 7, wherein the administering comprises intensity-modulated radiotherapy.

9. The method of claim 7, wherein the administering comprises emitting the radiation through a filter.

10. A method of providing radiation therapy to a patient in a corrected radiation dose distribution, the method comprising administering radiation to the patient in dose distribution that is substantially sombrero-shaped.

11. The method of claim 10, wherein the administering comprises intensity-modulated radiotherapy.

12. The method of claim 10, wherein the administering comprises emitting the radiation through a filter.

13. A method of shaping a radiation filter, the method comprising:
determining a target position variance for a radiation target;
producing a three-dimensional distribution model of the target position variance;
performing an inverse blurring operation on the intended dose distribution based on the three-dimensional distribution model to produce a corrected radiation dose distribution; and
shaping the radiation filter such that a radiation beam filtered by the radiation filter will produce substantially the corrected radiation dose distribution.

14. A radiation filter shaped according to the process of claim 13.

15. The radiation filter of claim 14, further comprising radiopaque material.

16. A radiation filter having a cross-sectional shape that defines an edge that is substantially sombrero-shaped.

17. A radiation filter kit comprising a plurality of radiation filters of different sizes, each of said radiation filters having a cross-sectional shape that defines an edge that is substantially sombrero-shaped.
